(19) United States
(12) Patent Application Publication
(10) Pub. No.: US 2026/0354767 A1
(43) Pub. Date: Nov. 12, 2026

(54) System and method for filtering motion artifacts from photoplethysmography signals

(71) Applicant: Senbiosys, Neuchâtel (CH)

(72) Inventors: Imane Ben M'Rad, Pully (CH); Clara Nguyen, Biel/Bienne (CH); Sandro Lecci, Echallens (CH); Assim Boukhayma, Neuchâtel (CH)

(21) Appl. No.: 19/664,417

(22) Filed: Apr. 30, 2026

Related U.S. Application Data
(60) Provisional application No. 63/802,019, filed on May 8, 2025.

(51) Int. Cl. A61B 5/00; A61B 5/024

(52) U.S. Cl. CPC A61B 5/7207; A61B 5/7264; A61B 5/02416

(57) ABSTRACT

Systems and methods are disclosed for filtering motion artifacts from photoplethysmography (PPG) signals to improve heart rate measurement accuracy. The system includes a PPG sensor that generates a raw PPG signal and a motion artifact filter that determines a perfusion index (PI) for the signal. The filter identifies individual peaks in the PPG signal, calculates a PI for each peak based on its AC and DC components, and excludes peaks with a PI exceeding a personalized threshold. This filtering reduces the influence of motion artifacts without relying on accelerometer data or computationally intensive algorithms. The filtered PPG signal is then used to compute health parameters such as heart rate, breathing rate, and heart rate variability. The invention offers improved accuracy and lower power consumption, making it suitable for integration into wearable devices.

### RELATED APPLICATIONS

This application claims the benefit under 35 USC 119(e) of U.S. Provisional Application No. 63/802,019, filed on May 8, 2025, which is incorporated herein by reference in its entirety.

### BACKGROUND OF THE INVENTION

Nowadays, wearable devices, such as fitness trackers or smartwatches, with optical heart rate sensors, are becoming common.

The technology behind these sensors is called photoplethysmography (PPG), which is a non-invasive optical measurement technique used to detect blood volume changes in living tissues such as the microvascular bed of the skin. PPG technology relies on the propagation of light into the skin. When the light diffuses into the tissue, a portion is absorbed at a level that fluctuates with the dilatation of the arterial vessels, induced by heart beats. A photodetector or sensor detects the light reflected from the tissue and transforms it into a photogenerated current. A PPG sensor requires a few optoelectronics components, such as a light source, e.g. light-emitting-diode (LED) to illuminate the living tissue, a photodetector (PD) to track any light intensity variation due to the blood volume change and an analog front-end (AFE) for signal conditioning and processing. Today, the importance of PPG for medical monitoring is proven by the number of primary vital signs directly or indirectly recordable out of it.

In one example, a PPG signal is obtained by shining light from the LED at a given wavelength, in the visible or near-infrared range, into a human tissue, e.g. finger, wrist, forehead, ear lobes. The PPG sensor or photodetector detects the light transmitted through (transmissive PPG) or reflected from (reflective PPG) the tissue and transforms it into a photogenerated current.

Motion artifacts in PPG signals refer to unwanted disturbances in the optical signal caused by physical movement of the sensor or the user. These artifacts introduce noise and distortions that interfere with accurate measurement of physiological parameters such as heart rate, heart rate variability (HRV), and blood oxygen saturation. Unlike physiological variability, motion artifacts are non-biological in origin and do not reflect actual changes in cardiovascular function, making them a significant challenge in wearable health monitoring.

In wearable devices—such as fitness trackers, smartwatches, and/or optical heart rate monitors—motion artifacts typically arise from several sources. The most common cause is relative motion between the sensor and the skin, which can temporarily alter light path geometry or sensor-to-skin contact pressure. Arm swing, wrist rotation, hand or finger movement or flexing, running, and even subtle vibrations from everyday movement can induce rapid changes in the reflected light signal. Additional factors such as sweat, loose device fit, or changes in ambient light can compound these effects, further degrading signal quality.

In particular, motion artifacts cause deflections of large amplitude in the PPG signal, impairing the monitoring of vital signs like cardiac cycles. This impact of motion artifacts on PPG signal fidelity is substantial, and, because the motion artifacts often overlap with the frequency range of the true pulse signal such as the heart rate (e.g., approximately 0.5–5 Hz), motion artifacts can mimic or obscure heartbeat-related waveforms. This leads to incorrect detection of pulse peaks, resulting in inaccurate heart rate readings or unreliable HRV metrics. In fitness and health applications, this can reduce user trust, impair activity classification, or compromise clinical decision-making. As a result, motion artifacts should be discarded or filtered from PPG signals, and effective motion artifact mitigation is a critical area of need in wearable health technology.

Perfusion Index (PI) is a physiological parameter derived from a PPG signal that provides a relative measure of the strength of blood flow at the sensor site. The variation of light absorption from tissue that determines a PPG signal is a small fraction of averaged reflected light from the tissue, resulting in a PPG signal with two components: a relatively large non-pulsatile (DC) component corresponding to the light diffusion through skin tissues and non-pulsatile blood layers, representing baseline absorption from static tissues and venous blood, and a relatively small pulsatile (AC) component, representing diffusion of the light through the arterial blood that causes the variation in arterial volume during the cardiac cycle. PI is calculated as the ratio of the pulsatile (AC) component of the PPG signal to the non-pulsatile (DC) component. Mathematically, the PI is typically expressed as PI = (AC / DC) × 100, yielding a dimensionless value indicative of peripheral perfusion. Higher PI values generally correspond to stronger arterial pulse signals, while lower values may indicate reduced blood flow or poor sensor contact. Typical PI values range from 0.5 to 5%, depending primarily on the temperature, skin tone and position of the hand (for measurements at the finger).

### SUMMARY OF THE INVENTION

Because the motion artifacts often overlap with the frequency range of a true pulse signal such as a heart rate, motion artifact noise cannot be separated from a photoplethysmography (PPG) signal using a bandpass filter. Additionally, some approaches to motion artifact mitigation that have been proposed rely on external motion sensors, complex signal transforms, or machine learning models, which can increase computational cost and power consumption.

At the same time, the present inventors have observed that movements that cause significant deflections and jumps in a PPG signal (causing motion artifacts) also result in a perfusion index (PI) above a physiological range, allowing motion artifacts to be distinguished from a true pulse signal using the PI even when the motion artifacts overlap with the frequency range of the true pulse signal.

Accordingly, the present invention relates to systems and methods for improving the accuracy of heart rate measurement from PPG signals by filtering motion artifacts using a PI-based criterion. The present disclosure introduces a lightweight, sensor-independent method that filters motion artifacts out of a PPG signal based on perfusion index by evaluating the reliability of individual peaks in the PPG signal based on their associated perfusion index. For each detected peak, the system calculates the AC and DC components of the PPG signal to derive a corresponding PI value. Peaks exhibiting a PI above a defined threshold—personalized for the user and/or the type of wearable device—are excluded from the heart rate computation. This selective exclusion enables more accurate heart rate estimation in the presence of motion without the need for accelerometer data or resource-intensive processing.

The proposed method offers advantages over existing solutions, including reduced computational complexity, elimination of dependency on auxiliary motion sensors, and improved adaptability across different users and devices. Experimental results demonstrate that the PI-based filtering technique significantly enhances heart rate measurement accuracy under motion conditions, making it particularly suitable for integration into power- and resource-constrained wearable platforms.

In general, according to one aspect, the invention features a system for filtering motion artifacts from PPG signals, the system comprising a PPG sensor and a computer system comprising a motion artifact filter. The PPG sensor generates a raw PPG signal, whereas the motion artifact filter receives the raw PPG signal, determines a perfusion index for the raw PPG signal, and generates a filtered PPG signal corresponding to the raw PPG signal by filtering the motion artifacts from the raw PPG signal based on the determined perfusion index and a predetermined filter threshold.

In embodiments, the motion artifact filter may determine the perfusion index for the raw PPG signal by detecting peaks in the raw PPG signal and determining a peak perfusion index for each of the detected peaks. The motion artifact filter may then filter the motion artifacts from the raw PPG signal based on a comparison of the peak perfusion index for each of the detected peaks to the predetermined filter threshold. In one example, the motion artifact filter may filter the motion artifacts from the raw PPG signal by excluding particular peaks of the detected peaks from the filtered PPG signal based on the comparison of the peak perfusion index for each of the detected peaks to the predetermined filter threshold (e.g., by excluding from the filtered PPG signal any of the detected peaks for which the peak perfusion index is greater than the predetermined filter threshold).

In one example, the predetermined filter threshold is approximately 1.2%. However, the predetermined filter threshold may vary and may be based on a current health measurement context, including an indication of a particular user for which the raw PPG signal was generated and/or an indication of a particular type of wearable housing for the PPG sensor.

In one exemplary configuration, a signal processor for the PPG sensor generates health measurement information based on the filtered PPG signal, including heart rate, breathing rate, heart rate variability, and/or peak-to-peak intervals for a user for which the raw PPG signal was generated, to list a few examples.

In general, according to another aspect, the invention features a method for filtering motion artifacts from PPG signals. A raw PPG signal is generated via a PPG sensor, and a perfusion index for the raw PPG signal is determined. A filtered PPG signal corresponding to the raw PPG signal is generated by filtering the motion artifacts from the raw PPG signal based on the determined perfusion index and a predetermined filter threshold.

In general, according to another aspect, the invention features an optical sensor system for generating health measurement information, the optical sensor system comprising a PPG sensor and a computer system, which comprises a signal processor. The PPG sensor comprises an optical emitter and an optical detector. The optical emitter transmits a light beam into tissue, the optical detector detects light from the tissue, and the PPG sensor generates a raw PPG signal based on the transmitted light beam and the detected light. The signal processor comprises a motion artifact filter that receives the raw PPG signal, determines a perfusion index for the raw PPG signal, and generates a filtered PPG signal corresponding to the raw PPG signal by filtering the motion artifacts from the raw PPG signal based on the determined perfusion index and a predetermined filter threshold. The signal processor then generates the health measurement information based on the filtered PPG signal.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a block diagram of an exemplary optical sensor system and signal processor according to an embodiment of the present invention;

FIG. 2 illustrates an example configuration of a PPG sensor in a wearable housing 112 of a wearable device along with the detected signal;

FIG. 3 shows an exemplary computer system that implements a signal processor, motion artifact filter, and/or filter threshold generator according to various embodiments of the present invention;

FIG. 4 schematically illustrates a signal processing pipeline using a wearable device according to an embodiment of the present invention;

FIG. 5 is a flow diagram of a method for filtering motion artifacts from a raw PPG signal according to an embodiment of the present invention;

FIG. 6 is a flow diagram of a method for filtering motion artifacts from a raw PPG signal according to a more detailed embodiment of the present invention;

FIG. 7 is a graph of a raw PPG signal showing a segment of stable resting data and a segment with motion artifacts caused by user movement;

FIG. 8 is a graph of a raw PPG signal and a filtered PPG signal showing how detected peaks identified as having perfusion index values above a filter threshold are excluded from a filtered signal as motion artifacts;

FIG. 9 is a graph of a raw PPG signal and a filtered PPG signal showing how detected peaks with PI values at or below a filter threshold are retained in the filtered PPG signal; and

FIG. 10 is a graph of a raw PPG signal and a filtered PPG signal showing a refined peak selection and inter-beat interval (IBI) cleaning process.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term "and/or" includes any and all combinations of one or more of the associated listed items. Also, all conjunctions used are to be understood in the most inclusive sense possible. Thus, the word "or" should be understood as having the definition of a logical "or" rather than that of a logical "exclusive or" unless the context clearly necessitates otherwise. Further, the singular forms and the articles "a", "an" and "the" are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

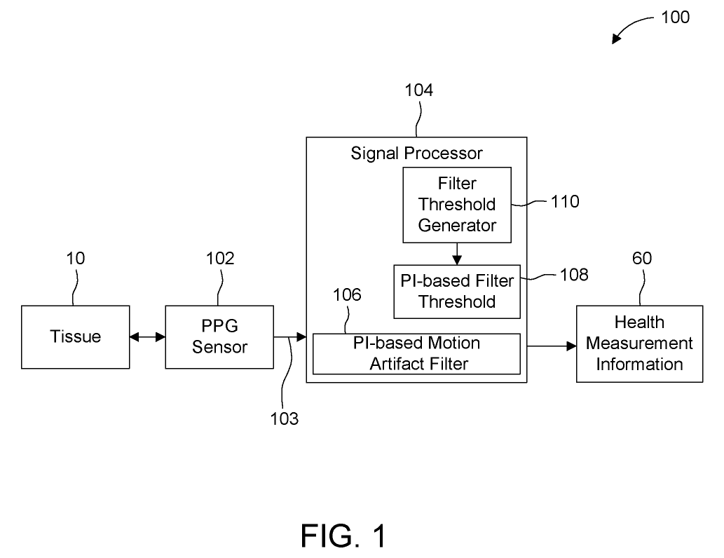

FIG. 1 is a diagram of an exemplary optical sensory system 100 generally comprising a photoplethysmography (PPG) sensor 102 and signal processor 104 according to the present invention.

The PPG sensor 102 generates a raw PPG signal 103 (e.g., based on light detected from tissue 10 via optical components of the PPG sensor 102), and the signal processor 104 receives the raw PPG signal 103 generated by the PPG sensor 102, processes the raw PPG signal, and generates health measurement information 60 based on the processing of the raw PPG signal (e.g., based on a filtered PPG signal).

In the illustrated example, the signal processor 104 comprises a motion artifact filter 106, which generates a filtered PPG signal based on the raw PPG signal, for example, by filtering motion artifacts from the raw PPG signal to produce the filtered PPG signal. The signal processor 104 may be configured to generate the health measurement information 60 based on the filtered PPG signal generated via the motion artifact filter 106.

More particularly, the motion artifact filter 106 determines a perfusion index (PI) for the raw PPG signal based on the raw PPG signal. The PI determined by the motion artifact filter 106 may represent a ratio of the pulsatile (AC) component of the raw PPG signal (generated via the PPG sensor 102) to the non-pulsatile (DC) component of that raw PPG signal, for example. In various embodiments, the motion artifact filter 106 may determine the PI for the raw PPG signal by detecting peaks in the raw PPG signal and determining a peak perfusion index for each of the detected peaks. The peak perfusion index for a particular peak detected in the raw PPG signal may represent a ratio of the pulsatile (AC) component to the non-pulsatile (DC) component of the raw PPG signal specific to that particular peak. The motion artifact filter 106 may be configured to continually or continuously (e.g., at predetermined time intervals, based on detected changes in the raw PPG signal) detect the peaks in the raw PPG signal and/or determine the peak perfusion index for each of these detected peaks, and the determined PI for the raw PPG signal may comprise a plurality of peak perfusion indexes, each corresponding to a particular peak detected in the raw PPG signal. The motion artifact filter 106 filters the motion artifacts from the raw PPG signal and/or generates the filtered PPG signal corresponding to the raw PPG signal based on the perfusion index determined for the raw PPG signal (including any peak perfusion indexes corresponding to peaks detected in the raw PPG signal).

The motion artifact filter 106 also filters the motion artifacts from the raw PPG signal and/or generates the filtered PPG signal corresponding to the raw PPG signal based on a filter threshold 108. More particularly, the motion artifact filter 106 filters the motion artifacts from the raw PPG signal and/or generates the filtered PPG signal based on a comparison of the determined perfusion index (including any peak perfusion indexes) to the filter threshold 108. In some embodiments, the motion artifact filter 106 filters the motion artifacts from the raw PPG signal based on a comparison of the peak perfusion index for each of the detected peaks in the raw PPG to the filter threshold 108, for example, by excluding particular peaks (of the detected peaks) from the filtered PPG signal based on the comparison of the peak perfusion index for each of the detected peaks to the filter threshold 108 (e.g., excluding any of the detected peaks for which the peak perfusion index is greater than the filter threshold).

The filter threshold 108 may be a value corresponding to and/or representing a delineation or boundary of an expected physiological range for determined or calculated perfusion index values. The expected physiological range may comprise a range of values for which a determined or calculated perfusion index value can be considered a valid representation of an actual physiological perfusion index condition of the tissue 10, as opposed to values outside of that range that can be assumed to be distorted or influenced by non-physiological factors such as motion artifacts such that the value may not be considered a valid representation of an actual physiological perfusion index condition of the tissue 10. In some embodiments, the filter threshold 108 may be a predetermined value and/or may be stored in memory accessible by the motion artifact filter 106. In some examples, the filter threshold 108 is a value in the range of 0.1% to 10%, or, in a particular example, approximately 1.2%.

In some embodiments, the filter threshold 108 may be a value that is determined based on a current health measurement context in such a way that the filter threshold 108 optimally and/or most accurately represents the boundary of the expected physiological range in that particular current health measurement context. The current health measurement context may include, for example, an indication of a particular user for which the raw PPG signal was generated and/or an indication of a particular type of wearable housing for the PPG sensor 102 that generated the raw PPG signal. For example, the filter threshold 108 could be tailored to individual users and/or different types of wearable devices (e.g., watch, ring) such that the filter threshold 108 tailored to that user and/or wearable type results in the most accurate filtering of the motion artifacts from the PPG signal when generating health measurement information 60 specific to that individual user and/or that type of wearable. Thus, the motion artifact filter 106 may be configured to filter the motion artifacts from the raw PPG signal based on different filter thresholds 108 corresponding to different health measurement contexts using, in any given health measurement context, the filter threshold 108 that is specific to and/or optimal for that current health measurement context.

In some embodiments, the motion artifact filter 106 may comprise and/or operate in conjunction with a filter threshold generator 110 that determines (e.g., calculates, selects) the filter threshold 108 based on the current health measurement context. In one example, the filter threshold generator 110 may be configured with a predetermined indication of the current health measurement context or may be configured to detect (e.g., based on current user data, sensor data, wearable type information data) the current health measurement. The filter threshold generator 110 may determine (e.g., calculate, select) a current filter threshold based on the current health measurement context and provide the determined current filter threshold to the motion artifact filter 106 as the filter threshold 108.

In some embodiments, the filter threshold generator 110 determines the filter threshold 108 for a particular user based on high-quality PPG signal data recorded during a period of time corresponding to an activity status and/or physiological state of that user designated as or known to be associated with more accurate PPG signals, such as a period of reduced user movement (e.g., when the user is sleeping). For example, during sleep, the user is typically in a steady state, and the perfusion index tends to reach its maximum under resting physiological conditions. The filter threshold generator 110 is configured to identify this period based on time of day, accelerometer data, health measurement information, or other contextual cues, and analyze the PPG signal during that period to calculate a personalized filter threshold 108 for that user that, for example, represents an upper bound of physiologically valid perfusion index values for that particular user. This sleep-based calibration enables the threshold to be tuned using optimal signal conditions, improving the reliability of subsequent artifact filtering during periods of activity or motion.

In various embodiments, the health measurement information 60 determined or generated based on the PPG signal may include one or more physiological or biometric parameters derived from characteristics of the filtered (e.g., filtered PPG signal from the motion artifact filter 106) or unfiltered PPG waveform. Examples of such health measurement information 60 include, but are not limited to, heart rate, heart rate variability (HRV), blood oxygen saturation (SpO₂), breathing rate, inter-beat intervals (IBI), perfusion index (PI), vascular age estimates, pulse transit time, and indicators of stress or autonomic nervous system activity. In certain implementations, health measurement information 60 may further include sleep stage classification, respiratory effort, or arrhythmia detection based on irregular pulse intervals or waveform morphology. The health measurement information 60 may be determined continuously or intermittently and may be used for real-time monitoring, trend analysis, or triggering alerts related to cardiovascular or respiratory health.

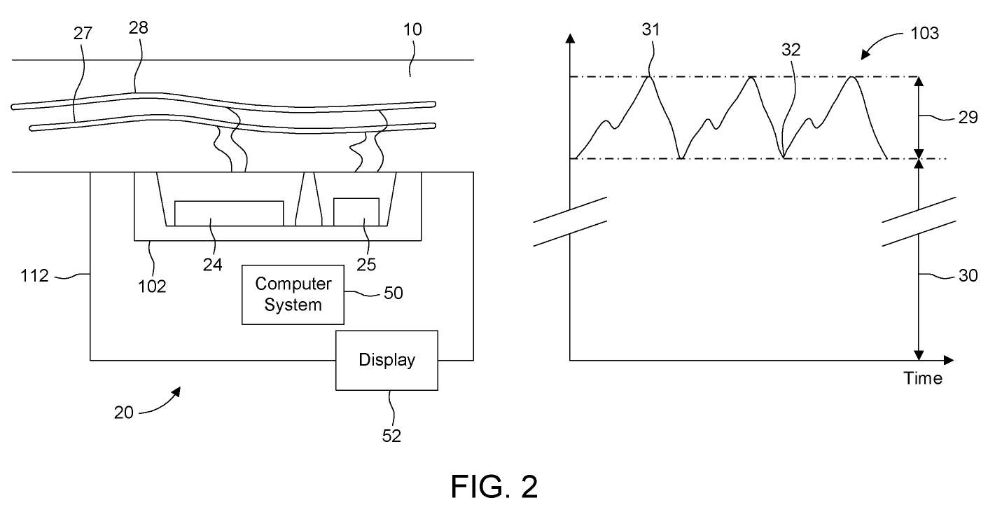

FIG. 2 illustrates an exemplary embodiment of a photoplethysmography (PPG) sensor 102 in a housing 112 of a wearable device 20. The sensor 102 is used to generate a raw PPG signal for processing by the signal processor 104 that is implemented in the device’s computer system 50 in one implementation. The PPG sensor 102 comprises an optical emitter 25 and an optical detector 24. The optical emitter 25 is configured to emit light into tissue 10 (e.g., skin) of a user, and the optical detector 24 is configured to detect light reflected from or transmitted through the tissue 10. In the illustrated example, the PPG sensor 102 operates in a reflective configuration, wherein the optical emitter 25 and the optical detector 24 are positioned on the same side of the tissue 10. However, in other embodiments, a transmissive configuration may be used, wherein the light passes through the tissue before being detected. The PPG sensor 102 is shown integrated into a wearable housing 112 of the wearable device 20, such as a wristband, ring, earbud, or other form factor configured to contact skin tissue 10 and measure changes in blood volume in the subcutaneous vasculature, including arterial vessels 27 and venous vessels 28.

Due to the pulsatile nature of arterial blood flow, light absorption in the tissue 10 varies over time, producing a raw PPG signal 103 comprising a pulsatile (AC) component 29 superimposed on a non-pulsatile (DC) component 30. The AC component 29 corresponds to periodic variations in arterial blood volume associated with the cardiac cycle, while the DC component 30 reflects baseline absorption from non-pulsatile blood and static tissue structures. The ratio of the AC component 29 to the DC component 30 defines a perfusion index (PI), which may be calculated globally or locally (e.g., for individual peaks) by the motion artifact filter 106 as described in connection with FIG. 1. In the illustrated signal waveform, reference numeral 31 denotes a systolic point (peak) of the PPG signal, and reference numeral 32 denotes a diastolic point (trough). These periodic features of the raw PPG signal may be used by the signal processor 104 to detect pulse peaks and to calculate physiological parameters such as heart rate, heart rate variability, and other health measurement information 60, which can be presented to the user via an integrated display 52, in some examples.

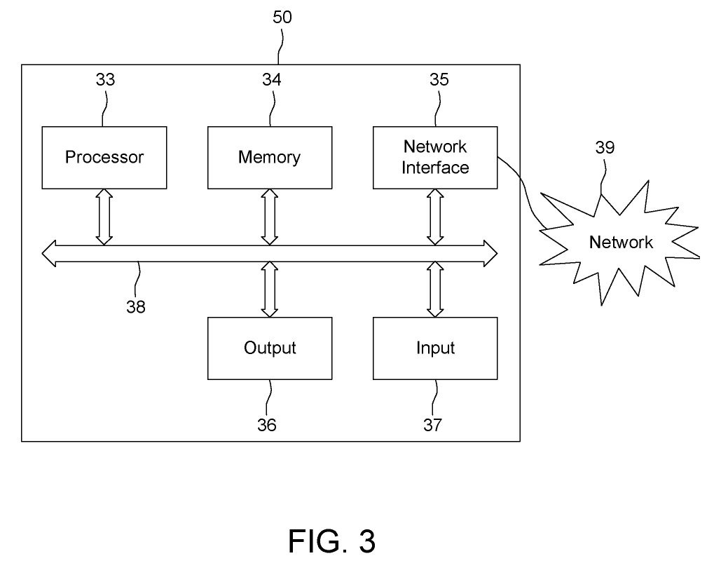

FIG. 3 illustrates an exemplary computer system 50 that may be used to implement one or more components and processes described in connection with the optical sensor system 100 and signal processor 104 of FIG. 1. In particular, the computer system 50 corresponds to and implements the signal processor 104 in one example, and may be configured to perform processing operations for generating health measurement information 60 based on a raw PPG signal received from the PPG sensor 102. In some embodiments, the computer system 50 may be integrated within the wearable housing 112 of the device 20, such as a wristband, ring, or earbud, or may be communicatively coupled to such a wearable device via a wired or wireless connection.

The computer system 50 includes a processor 33, memory 34, a data bus 38, and a network interface 35. The processor 33 may include one or more general-purpose microprocessors, central processing units (CPUs), digital signal processors (DSPs), field-programmable gate arrays (FPGAs), or application-specific integrated circuits (ASICs), and may be configured to execute instructions for implementing the signal processor 104 functionality, including operations performed by the motion artifact filter 106 and the filter threshold generator 110 described in FIG. 1. The memory 34 may include both volatile and non-volatile memory, such as random-access memory (RAM), read-only memory (ROM), flash memory, and other types of static or dynamic memory for storing program instructions, filter thresholds 108, intermediate values (e.g., perfusion index calculations), and health measurement information 60.

The data bus 38 facilitates communication between the processor 33, memory 34, network interface 35, and any optional input/output interfaces. The network interface 35 may enable the computer system 50 to communicate with an external network 39, such as a wireless network or the Internet, for purposes such as remote data upload, cloud-based processing, firmware updates, or synchronization with companion applications. In some implementations, the computer system 50 may be coupled to output components, such as the integrated display 52 or other display, for presenting health measurement information 60, or to input devices for user interaction.

Through execution of program instructions stored in memory 34, the processor 33 is configured to receive a raw PPG signal 103 from the PPG sensor 102, determine a perfusion index (e.g., including peak perfusion indexes), compare the perfusion index values to a filter threshold 108, and generate a filtered PPG signal from which health measurement information 60 may be derived.

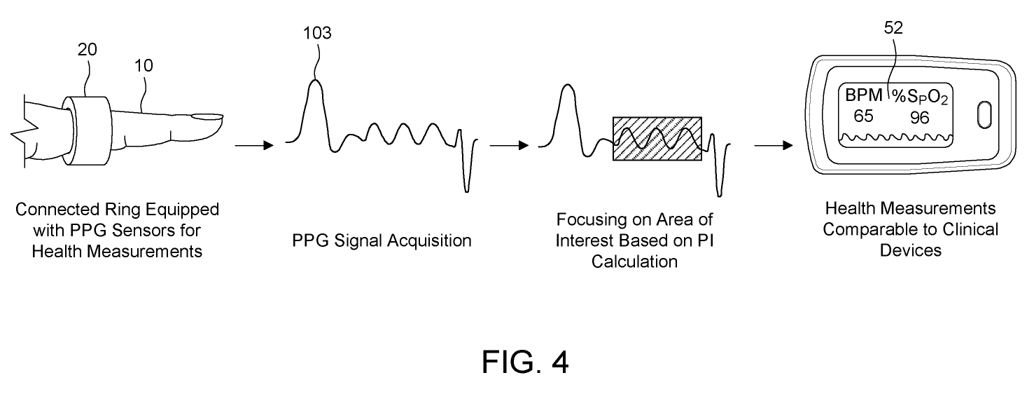

FIG. 4 illustrates an exemplary overview of a processing pipeline for generating health measurement information 60 using the optical sensor system 100 described in FIG. 1. As shown, a wearable device 20, such as a connected ring, is equipped with a PPG sensor 102 including an optical emitter 25 and optical detector 24 in contact with tissue 10 of the user (e.g., the skin of a finger). The PPG sensor 102 emits light into the tissue and detects reflected light to generate a raw PPG signal corresponding to blood volume changes within underlying vessels 27 and 28, as previously described in connection with FIG. 2.

The raw PPG signal 103 is then acquired and transmitted to the signal processor 104 implemented on the computer system 50 for analysis. As described with respect to FIG. 1, the signal processor 104 includes a motion artifact filter 106 that evaluates the raw PPG signal to determine a perfusion index (PI), which may be calculated globally or per-peak using the AC component 29 and DC component 30 of the signal. Based on the PI, the motion artifact filter 106 identifies and isolates an area of interest within the raw PPG signal that falls within an expected physiological range, excluding peaks or segments associated with motion artifacts (e.g., peaks exceeding the filter threshold 108). This selective filtering results in a filtered PPG signal with reduced noise and distortion.

Using the filtered PPG signal, the signal processor 104 generates health measurement information 60 presented in a display device 52, which may include, for example, heart rate, blood oxygen saturation (SpO₂), and other physiological parameters. As shown in the final portion of FIG. 4, the filtered output enables accurate health measurements that are comparable in reliability to those obtained from clinical-grade monitoring devices, thereby enhancing the performance of wearable devices while maintaining low computational and power requirements.

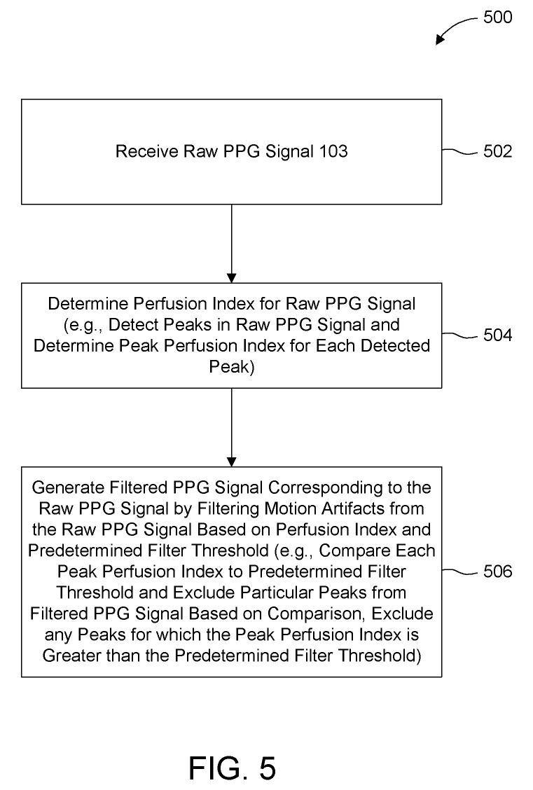

FIG. 5 is a flow diagram illustrating an exemplary general method 500 for filtering motion artifacts from a photoplethysmography (PPG) signal, in accordance with various embodiments of the present invention. The method 500 may be performed, for example, by the signal processor 104 described in connection with FIG. 1 as implemented on the computer system 50 of FIGS. 2 and 3, including the motion artifact filter 106 and the optional filter threshold generator 110. The method 500 comprises three primary steps—502, 504, and 506.

At step 502, the method comprises generating a raw PPG signal 103 via a PPG sensor, such as PPG sensor 102, which includes an optical emitter 25 and an optical detector 24 in contact with tissue 10. The raw PPG signal corresponds to variations in light intensity reflected from or transmitted through the tissue due to pulsatile blood flow, as described in connection with FIGS. 1 and 2. The raw PPG signal may include both a pulsatile (AC) component and a non-pulsatile (DC) component.

At step 504, the method comprises determining a perfusion index for the raw PPG signal 103. In various embodiments, this includes detecting peaks in the raw PPG signal and determining a peak perfusion index for each detected peak. The peak perfusion index may be calculated as the ratio of the AC component to the DC component of the raw PPG signal at the time of each detected peak. As described with respect to the motion artifact filter 106, this step may be performed continuously or intermittently and may include generating a set of peak perfusion index values corresponding to the detected peaks in the raw signal.

At step 506, the method comprises generating a filtered PPG signal by filtering the motion artifacts from the raw PPG signal based on the determined perfusion index and a predetermined filter threshold 108. This includes comparing the peak perfusion index for each detected peak to the filter threshold 108 and excluding from the filtered PPG signal any peaks for which the peak perfusion index exceeds the threshold. The filter threshold may be a fixed value (e.g., approximately 1.2%) or may be determined dynamically based on a current health measurement context, such as a particular user profile or wearable form factor. In some embodiments, the threshold may be determined by the filter threshold generator 110. The resulting filtered PPG signal may then be used to generate health measurement information 60, such as heart rate, heart rate variability, blood oxygen saturation, or other physiological metrics.

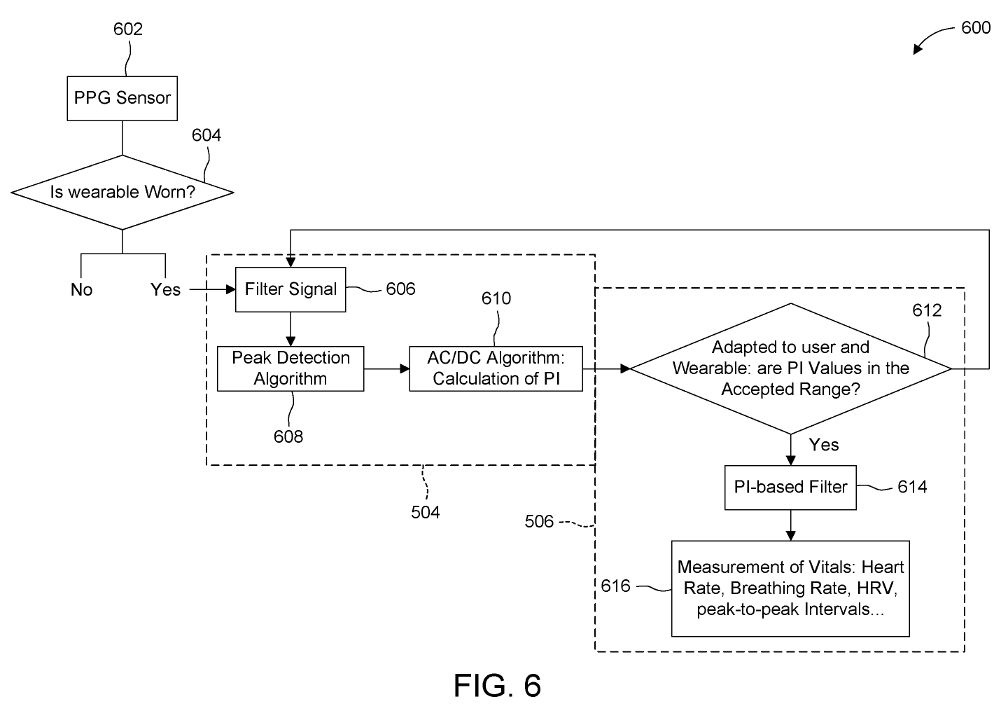

FIG. 6 is a more detailed flow diagram illustrating an example method 600 for processing a photoplethysmography (PPG) signal to generate health measurement information 60, using a perfusion index (PI)-based filtering approach. The method 600 may be implemented, for example, by the signal processor 104 of FIG. 1, which includes a motion artifact filter 106 and may optionally include a filter threshold generator 110. The steps of the method 600 reflect a practical embodiment of the PI-based motion artifact filtering technique described herein.

At step 602, a PPG sensor 102 (e.g., comprising optical emitter 25 and optical detector 24) generates a raw PPG signal by emitting light into tissue 10 and detecting reflected light corresponding to variations in blood volume. At decision step 604, the computer system 50 determines whether the wearable device containing the PPG sensor 102 is currently being worn. If the wearable is not worn, the method may pause or restart. If the wearable is worn, the method continues to step 606.

Breaking down step 504 from FIG. 5, at step 606, the computer system 50 implements the signal processor 104 that performs initial signal filtering to suppress noise or irrelevant frequency components. At step 608, the signal processor 104 executes a peak detection algorithm to identify individual peaks within the raw PPG signal, each corresponding to a cardiac cycle. At step 610, the motion artifact filter 106 performs an AC/DC analysis to calculate a perfusion index (PI) for each detected peak. As described in connection with FIG. 2, the PI is computed as the ratio of the pulsatile (AC) component 29 to the non-pulsatile (DC) component 30 of the PPG signal.

At decision step 612, the motion artifact filter 106 evaluates whether the calculated PI values for the detected peaks fall within an accepted physiological range. This evaluation may be based on a predetermined filter threshold 108 that is static or dynamically adapted to a current health measurement context such as a particular user or wearable housing type (e.g., as determined by filter threshold generator 110). If the PI values fall outside the accepted range, indicating likely motion artifacts, the method may return to an earlier stage for reprocessing. In one example, step 612 may be repeated continuously or continually for each detected peak in the raw PPG signal searching for peak for the corresponding PI fall within an accepted range.

At step 614, a PI-based filter is applied when the PI is acceptable, i.e., neither too high and/or too low. Here, any peaks with PI values determined in step 612 to fall outside the accepted range may be excluded from the filtered PPG signal, while any peaks with PI values determined in step 612 to fall within the accepted range may be included as part of the filtered PPG signal. Thus, the filter may exclude from further analysis any detected peaks associated with a PI exceeding the filter threshold 108. This filtering step results in a refined, motion-artifact-reduced version of the PPG signal.

At step 616, the filtered PPG signal is used by the signal processor 104 implemented by the computer system 50 to generate health measurement information 60. This may include, for example, heart rate, breathing rate, heart rate variability (HRV), and peak-to-peak intervals. As described above, these health metrics may be used for real-time monitoring, trend analysis, or triggering alerts in wearable devices such as smartwatches, rings, or earbuds.

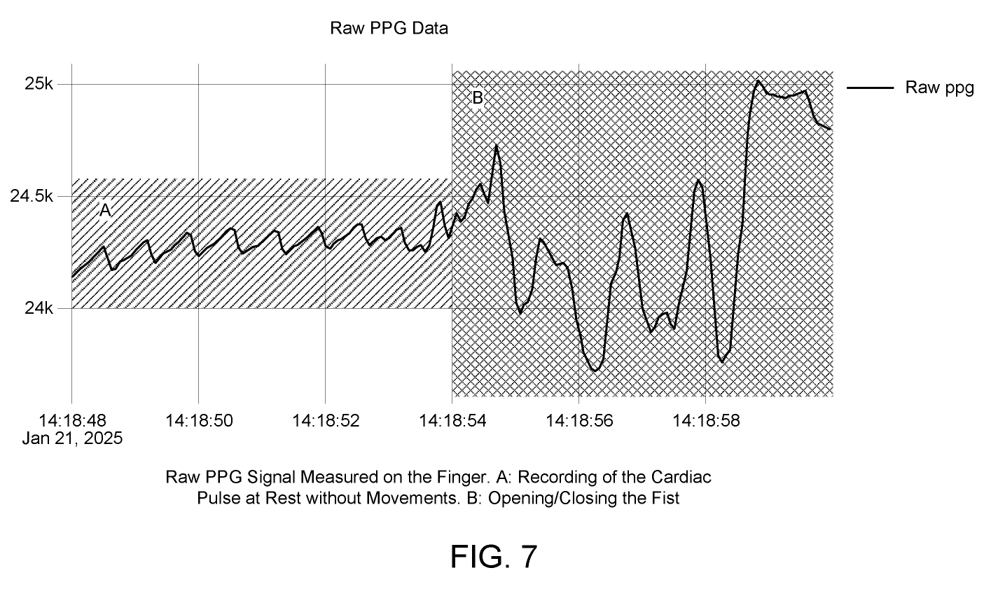

FIG. 7 is a graph illustrating an example of a raw photoplethysmography (PPG) signal generated by the PPG sensor 102 (e.g., comprising optical emitter 25 and optical detector 24) of the optical sensor system 100, as described in FIGS. 1 and 2. The signal shown in FIG. 7 is measured from the tissue 10 of a user's finger using a wearable device configured with the PPG sensor 102. The vertical axis represents signal amplitude (in arbitrary units), while the horizontal axis represents time. The graph includes two highlighted regions labeled A and B, each demonstrating a distinct condition during signal acquisition.

Region A represents a segment of the raw PPG signal recorded while the user was at rest, with the finger remaining stationary. As shown, the signal in this region displays consistent, periodic waveform morphology characteristic of an undistorted cardiac pulse. In this condition, the raw PPG signal includes a clearly identifiable pulsatile (AC) component superimposed on a relatively stable non-pulsatile (DC) baseline, which would allow accurate calculation of a perfusion index (PI) as described with reference to the motion artifact filter 106.

Region B represents a segment of the raw PPG signal during which the user performed an active movement (e.g., opening and closing the fist), simulating a common source of motion artifacts in wearable health monitoring. As shown in the waveform, this region is marked by significant deflections and irregular signal morphology. The signal includes abrupt changes in amplitude and frequency, as well as large, erratic fluctuations inconsistent with the expected shape of a physiological pulse. These distortions make accurate pulse detection and health measurement challenging and would typically lead to incorrect calculations of heart rate or other biometric parameters if left unfiltered.

This figure demonstrates the practical need for filtering motion artifacts from the raw PPG signal based on a perfusion index threshold, as described in the context of the motion artifact filter 106. Peaks in region B are likely to yield abnormally high PI values that fall outside the accepted physiological range defined by the filter threshold 108. Using the PI-based filtering approach disclosed herein, the system can identify and exclude such peaks from the filtered PPG signal, thereby preserving the accuracy of health measurement information 60 (e.g., heart rate, heart rate variability, or blood oxygen saturation) derived from motion-free segments such as region A.

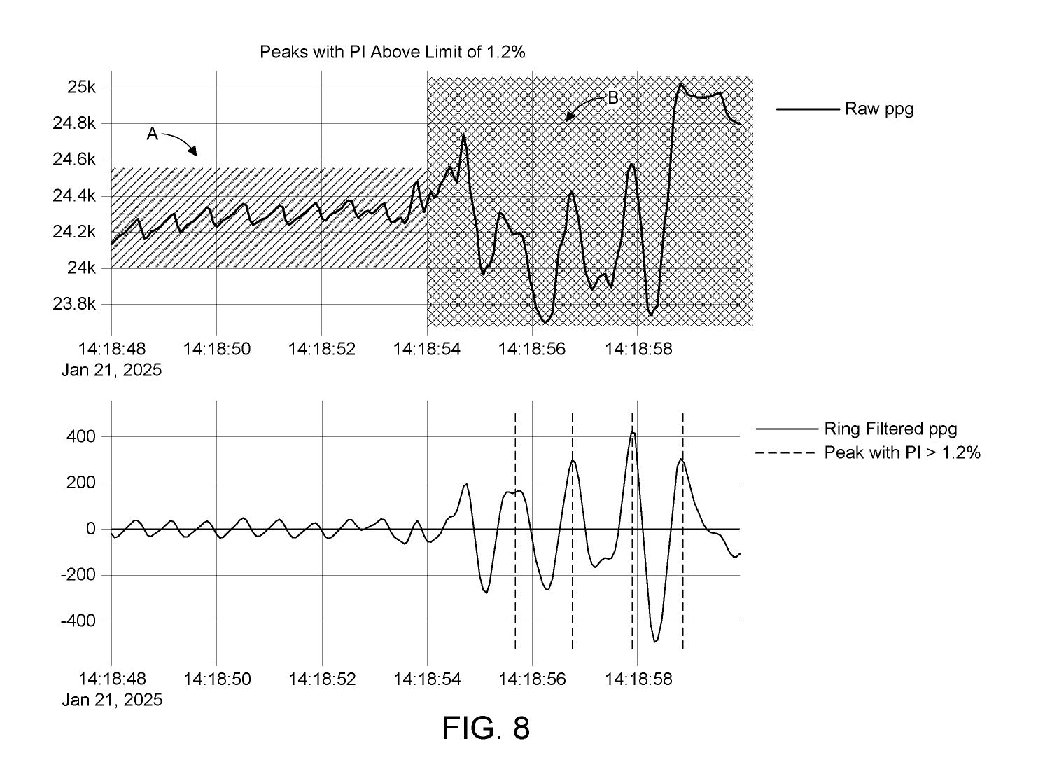

FIG. 8 illustrates an example of motion artifact filtering based on a perfusion index (PI) threshold, as applied to a raw PPG signal generated by a PPG sensor 102 (e.g., including optical emitter 25 and optical detector 24) in contact with tissue 10, as described in FIGS. 1 and 2. The figure includes two plots: an upper plot showing the unprocessed raw PPG signal and a lower plot showing the same signal after processing and peak-based PI filtering.

In the upper plot, the raw PPG signal is recorded from the tissue 10 of a user's finger using a wearable device configured with the optical sensor system 100. This signal includes two regions, corresponding to those previously shown and described in FIG. 7. Region A reflects a period of rest, during which the signal exhibits periodic, stable cardiac pulse morphology. Region B reflects a period during which the user is actively moving (e.g., opening and closing the fist), resulting in signal distortion due to motion artifacts. These motion artifacts appear as large, irregular deflections in the waveform.

In the lower plot, the signal is shown after processing by the signal processor 104, including the motion artifact filter 106. The processed signal represents the filtered PPG signal, where each peak has been evaluated using a per-peak PI calculation. Peaks for which the PI exceeds a threshold value of 1.2%—which may be a predefined or dynamically determined filter threshold 108—are indicated by vertical dashed lines. These peaks are identified by the motion artifact filter 106 as likely corresponding to motion-corrupted data and are excluded from the filtered PPG signal.

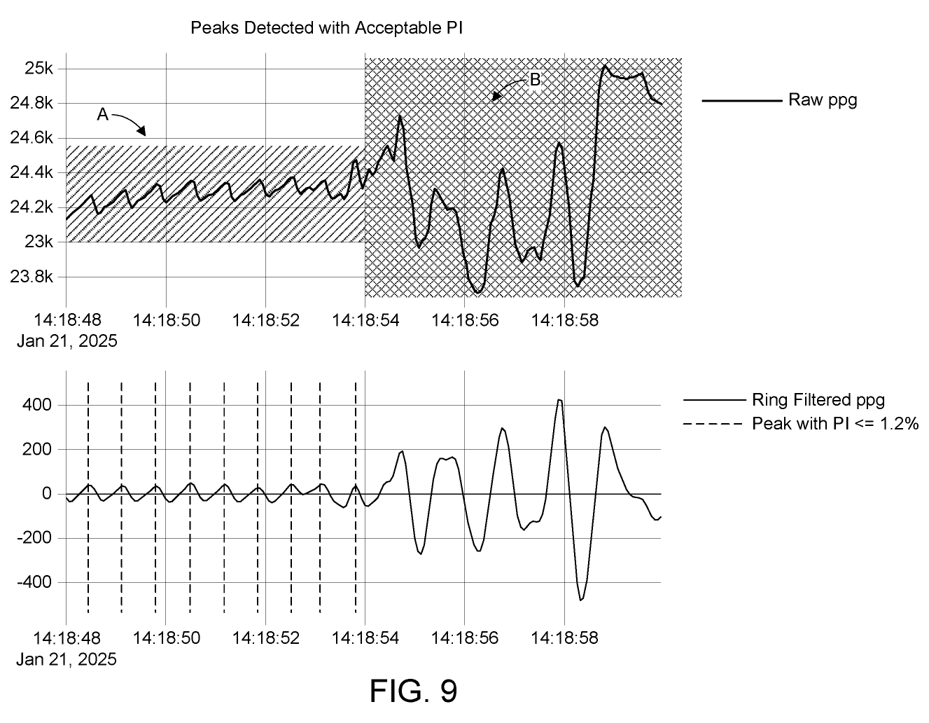

FIG. 9 illustrates the detection and identification of peaks within a photoplethysmography (PPG) signal for which the perfusion index (PI) falls within an acceptable physiological range, in accordance with the methods described herein. As in prior figures, the PPG signal shown in FIG. 9 is generated by the PPG sensor 102, including optical emitter 25 and optical detector 24, positioned in contact with tissue 10 of a user, for example via a wearable device such as a ring or wristband (see FIG. 1).

The upper plot in FIG. 9 shows the raw PPG signal, comprising two distinct regions: region A, representing a period of rest, and region B, representing a period of motion (e.g., fist opening and closing). These regions are consistent with those shown and described in FIGS. 7 and 8. Despite the presence of motion in region B, some peaks within both regions may still correspond to physiologically valid heartbeats if their associated PI values remain within a threshold range.

The lower plot in FIG. 9 shows the filtered PPG signal following signal processing by the signal processor 104, and more specifically, the motion artifact filter 106. Here, the filter performs peak detection and calculates a peak PI for each detected peak using the AC and DC components of the signal. Peaks for which the PI is less than or equal to the filter threshold—e.g., 1.2%—are indicated by vertical dashed black lines. These peaks are retained in the filtered PPG signal for subsequent physiological analysis.

This figure complements FIG. 8 by demonstrating how the filtering method selectively retains only those peaks with PI values that fall within a valid physiological range, as determined by comparison to a predetermined or contextually adapted filter threshold 108 (e.g., defined by filter threshold generator 110). Unlike FIG. 8, which shows the exclusion of high-PI peaks, FIG. 9 confirms that the algorithm allows valid peaks to pass through the filter, preserving data integrity for reliable health measurement information 60.

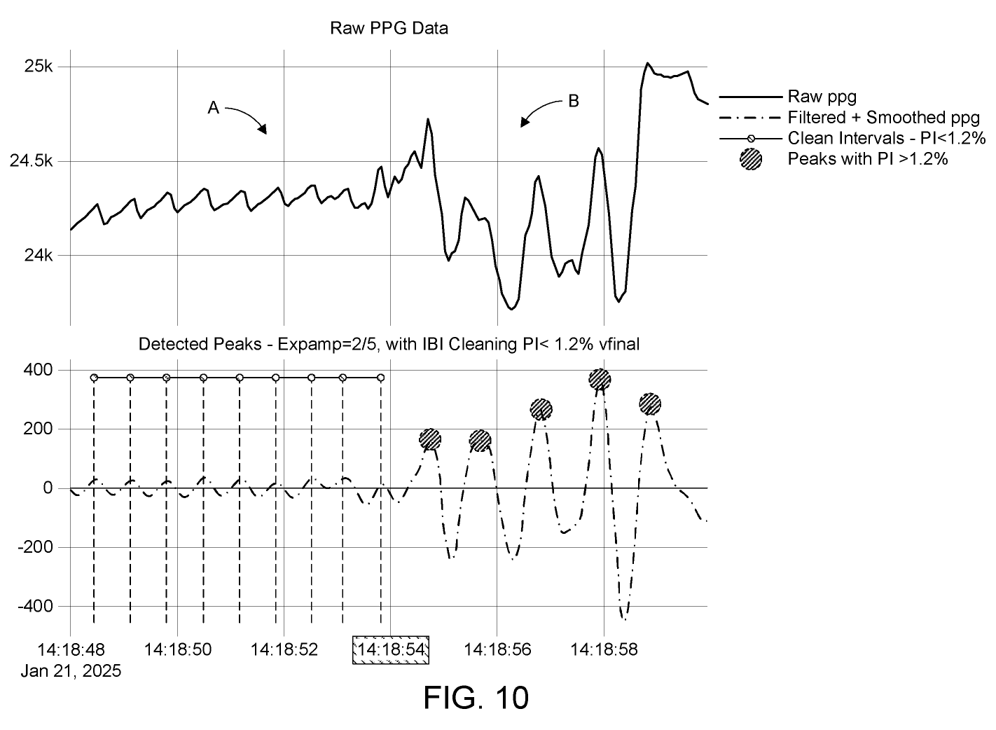

FIG. 10 illustrates an example of peak selection and interval cleaning based on per-peak perfusion index (PI) analysis, as applied to a raw photoplethysmography (PPG) signal acquired from tissue 10 using the PPG sensor 102 (e.g., comprising optical emitter 25 and optical detector 24), as described in connection with FIGS. 1 and 2. The figure includes two plots—an upper plot showing the raw PPG signal and a lower plot showing the filtered and smoothed PPG signal, along with peak annotations based on PI values.

In the upper plot, as before, the raw PPG signal includes region A, representing a resting phase with stable cardiac waveforms, and region B, representing a motion phase during which the user performed a physical action such as opening and closing the fist. As described previously with reference to FIGS. 7 through 9, the motion in region B introduces signal deflections indicative of motion artifacts.

The lower plot displays the same signal after processing by the signal processor 104, including motion artifact filtering and smoothing by the motion artifact filter 106. The curve represents the filtered PPG signal. Dashed vertical lines indicate peaks that were successfully detected and retained following PI-based evaluation and filtering. As shown, intervals associated with PI values less than or equal to the threshold (e.g., 1.2%) are retained as “clean intervals,” while peaks with PI values greater than the threshold are highlighted with dashed circular callouts.

This figure demonstrates a combination of steps as shown in FIG. 6, including peak detection (step 608), PI calculation (step 610), threshold comparison (step 612), and filtering (step 614). Specifically, the method identifies a subset of detected peaks within the raw PPG signal whose associated PI values fall within a valid physiological range. Peaks with PI values greater than the filter threshold 108 are excluded or flagged, allowing only valid intervals to be used for further physiological analysis.

The result of this process is a refined set of inter-beat intervals (IBIs), which can then be used to generate accurate health measurement information 60 such as heart rate, HRV, and peak-to-peak intervals. By isolating only the clean signal segments and excluding high-PI peaks, the system avoids false detections due to motion artifacts and ensures that derived biometric metrics reflect true cardiovascular activity. FIG. 10 thus provides visual support for the filtering and selection methods described in FIGS. 1, 5, and 6, further illustrating how the system improves measurement reliability in real-world wearable scenarios.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

While this invention has been particularly shown and described with references to preferred embodiments thereof, it will be understood by those skilled in the art that various changes in form and details may be made therein without departing from the scope of the invention encompassed by the appended claims.

What is claimed is:

1. A system for filtering motion artifacts from photoplethysmography (PPG) signals, the system comprising:
a PPG sensor for generating a raw PPG signal; and
a computer system comprising a motion artifact filter that receives the raw PPG signal, determines a perfusion index for the raw PPG signal, and generates a filtered PPG signal corresponding to the raw PPG signal by filtering the motion artifacts from the raw PPG signal based on the determined perfusion index and a predetermined filter threshold.

2. The system of claim 1, wherein the motion artifact filter determines the perfusion index for the raw PPG signal by detecting peaks in the raw PPG signal and determining a peak perfusion index for each of the detected peaks.

3. The system of claim 2, wherein the motion artifact filter filters the motion artifacts from the raw PPG signal based on a comparison of the peak perfusion index for each of the detected peaks to the predetermined filter threshold.

4. The system of claim 3, wherein the motion artifact filter filters the motion artifacts from the raw PPG signal by excluding particular peaks of the detected peaks from the filtered PPG signal based on the comparison of the peak perfusion index for each of the detected peaks to the predetermined filter threshold.

5. The system of claim 4, wherein the motion artifact filter filters the motion artifacts from the raw PPG signal by excluding from the filtered PPG signal any of the detected peaks for which the peak perfusion index is greater than the predetermined filter threshold.

6. The system of claim 1, wherein the predetermined filter threshold is approximately 1.2%.

7. The system of claim 1, wherein the predetermined filter threshold is based on a current health measurement context and/or was calculated during a period of inactivity.

8. The system of claim 7, wherein the current health measurement context includes an indication of a particular user for which the raw PPG signal was generated and/or an indication of a particular type of wearable housing for the PPG sensor.

9. The system of claim 1, wherein a signal processor for the PPG sensor generates health measurement information based on the filtered PPG signal.

10. The system of claim 9, wherein the health measurement information includes heart rate, breathing rate, heart rate variability, and/or peak-to-peak intervals for a user for which the raw PPG signal was generated.

11. A method for filtering motion artifacts from photoplethysmography (PPG) signals, the method comprising:
generating a raw PPG signal via a PPG sensor;
determining a perfusion index for the raw PPG signal; and
generating a filtered PPG signal corresponding to the raw PPG signal by filtering the motion artifacts from the raw PPG signal based on the determined perfusion index and a predetermined filter threshold.

12. The method of claim 11, further comprising determining a perfusion index for the raw PPG signal by detecting peaks in the raw PPG signal and determining a peak perfusion index for each of the detected peaks.

13. The method of claim 12, further comprising filtering the motion artifacts from the raw PPG signal based on a comparison of the peak perfusion index for each of the detected peaks to the predetermined filter threshold.

14. The method of claim 13, further comprising filtering the motion artifacts from the raw PPG signal by excluding particular peaks of the detected peaks from the filtered PPG signal based on the comparison of the peak perfusion index for each of the detected peaks to the predetermined filter threshold.

15. The method of claim 14, further comprising filtering the motion artifacts from the raw PPG signal by excluding from the filtered PPG signal any of the detected peaks for which the peak perfusion index is greater than the predetermined filter threshold.

16. The method of claim 11, wherein the predetermined filter threshold is approximately 1.2%.

17. The method of claim 11, wherein the predetermined filter threshold is based on a current health measurement context and/or was calculated during a period of inactivity.

18. The method of claim 17, wherein the current health measurement context includes an indication of a particular user for which the raw PPG signal was generated and/or an indication of a particular type of wearable housing for the PPG sensor.

19. The method of claim 18, further comprising generating health measurement information based on the filtered PPG signal.

20. An optical sensor system for generating health measurement information, the optical sensor system comprising:
a photoplethysmography (PPG) sensor comprising an optical emitter and an optical detector, wherein the optical emitter transmits a light beam into tissue, the optical emitter detects light from the tissue, and the PPG sensor generates a raw PPG signal based on the transmitted light beam and the detected light; and
a computer system comprising a signal processor for generating the health measurement information, the signal processor comprising a motion artifact filter that receives the raw PPG signal, determines a perfusion index for the raw PPG signal, and generates a filtered PPG signal corresponding to the raw PPG signal by filtering the motion artifacts from the raw PPG signal based on the determined perfusion index and a predetermined filter threshold, wherein the signal processor generates the health measurement information based on the filtered PPG signal.
